(19) United States
(12) Patent Application Publication
(10) Pub. No.: US 2017/0134844 A1
(43) Pub. Date: May 11, 2017

(54) IN-EAR MONITOR MANUFACTURING PROCESS

(71) Applicant: Logitech Europe S.A, Lausanne (CH)

(72) Inventors: Vincent LIU, Irvine, CA (US); Phillippe DEPALLENS, San Clemente, CA (US); Todd William LANSINGER, Irvine, CA (US); Joseph A. SAGGIO, JR., Anaheim Hills, CA (US)

(21) Appl. No.: 15/406,658

(22) Filed: Jan. 13, 2017

Related U.S. Application Data
(63) Continuation-in-part of application No. 15/275,004, filed on Sep. 23, 2016.
Application No. 15/275,004 is a reissue of application No. 14/061,885, filed on Oct. 24, 2013, now Pat. No. 9,042,589.

(51) Int. Cl. H04R 1/10; A61B 5/00; A61B 5/107

(52) U.S. Cl. CPC H04R 1/1016; A61B 5/1076; A61B 5/1078; A61B 5/1079; A61B 5/6817; H04R 1/1075; A61B 2503/12; H04R 2460/15

(57) ABSTRACT

The present disclosure generally provides a method of manufacture of a custom fit in-ear module, including capturing an anatomical representation of a body part at a first location; transferring the stored data to a second electronic device positioned at a second location; forming a three-dimensional digital model from the stored data using the second electronic device; transforming the three-dimensional digital model, wherein the transforming comprises forming a cavity within the three-dimensional digital model, wherein the cavity is sized to receive an acoustic output member and one or more drivers; transferring the transformed three dimensional model to a third electronic device positioned at a third location; forming a body of an in-ear monitor using the transformed three dimensional model; and positioning the acoustic output member and one or more drivers within the formed body, wherein the acoustic output member and one or more drivers reside at least partially within the cavity.

### CROSS-REFERENCE TO RELATED APPLICATIONS

This application is a continuation-in-part of application Ser. No. 15/275,004, which is an application for reissue of U.S. Pat. No. 9,042,589. This application is hereby incorporated herein by reference.

### BACKGROUND

Field

Embodiments of the present disclosure generally relate to a process for manufacturing a custom fit in-ear monitor.

Background

### DESCRIPTION OF THE RELATED ART

In-ear monitors provide an enhanced listening experience for studio recording, stage performance, and audiophile listening. To listen to recorded music, in-ear monitors may be hard-wired or wirelessly connected to a music player to listen to recorded music. For performance or recording of live music, in-ear monitors may be connected directly or wirelessly to a receiver pack worn by the user (e.g., mammal) or connected directly to a transmitter such as a mixer or amplifier.

In-ear monitors are superior to loudspeakers in that they facilitate a personalized mix of audio sources. In-ear monitors may reduce, eliminate or control ambient noise, including crowd and stage noise. In-ear monitors also improve the clarity of the combined mix, or “monitor mix,” of the performers' voices, instruments and/or music tracks in order for the performers to hear other pertinent audio during a performance at a venue.

In-ear monitors generally comprise a shell, or a case that contacts the external ear canal of the end user, and a driver assembly, which includes the drivers, crossover circuit, and other relevant hardware. In-ear monitors may be generic in size and shape, or they may be customized to fit the end user. An intermediate alternative is to sell generic in-ear monitors with removable and replaceable ear tips, such that the end user may choose from a selection of ear tips of varying size, shape and color to partially customize the in-ear monitor. Generic in-ear monitors, which are not manufactured to fit a specific user's ears, have several disadvantages. Generic in-ear monitors tend to be less comfortable to the user because they do not account for differences in individual ear shape. Also, without customization, it is very difficult to design a generic in-ear monitor that can be comfortably inserted into the external ear canal. Therefore, generic in-ear monitors tend to be shallow in design and fit in the outer ear without penetrating the external ear canal. As a result of the shallow design, there is space between the end of the in-ear monitor and the eardrum, resulting in poor isolation and poor sound quality. Finally, a generic in-ear monitor often contains only a single driver, which provides a sub-optimal listening experience.

A more fully customized in-ear monitor improves the listening experience in several ways. Positioning the in-ear monitor near to, but a safe distance from, the eardrum serves to enhance the quality of sound. A closer fit within the end user's ear canal limits movement during the listening experience and improves noise isolation, which both also enhance the quality of sound received by the user. The tailored shape of a customized in-ear monitor may also improve the experience of inserting and removing the device, as a technician may design the body of the in-ear monitor such that insertion and removal are simplified. Customized in-ear monitors may include two, three, or more drivers, which improves the quality of sound provided to the user.

A common process for manufacturing custom in-ear monitors may include the following steps. First, measurements of the end user's external ear canal are taken, for example by using a wax mold. A specialist/technician then reviews and refines the wax mold to create a revised model that represents an approximation of the external shape and dimensions of the in-ear monitor to provide a close fit. The internal shape and dimensions of the molded in-ear monitor are then manually tailored to accommodate the required electrical components and other hardware. The revised shape is then used to fabricate the custom in-ear monitor body or shell. The electrical and other hardware components are then inserted into the custom in-ear monitor shell to form the complete device.

Because the effectiveness of the in-ear monitor depends on the accuracy and precision of the wax mold, the wax mold process is specialized and must be performed by a skilled technician. Further, the wax molding process must be completed at a special location where the technician's materials and equipment reside. As a result, an end user may be required to visit a specialized lab at which the wax mold is taken. Such a visit may require traveling long distances and waiting extended periods of time for the steps of the process to be completed.

Therefore, there is a need for a simplified and convenient process for manufacturing in-ear monitors that overcomes the inefficiencies identified above and improves the comfort and sound quality of the formed custom in-ear monitor.

### SUMMARY

Embodiments of the present disclosure generally relate to a process of forming a custom in-ear monitor that includes capturing a digital anatomical representation of a surface of a body part at a first location, wherein capturing comprises digitally scanning at least a portion of the body part and storing data associated with the captured surface dimensions of the body part in non-volatile memory of a first electronic device, transferring the stored data to a second electronic device positioned at a second location, forming a three-dimensional digital model from the stored data using the second electronic device, transforming the three-dimensional digital model, transferring the transformed three dimensional model to a third electronic device positioned at a third location, forming, at the third location, a body of an in-ear monitor using the transformed three dimensional model, and positioning the acoustic output member and one or more drivers within the formed body, wherein the acoustic output member and one or more drivers reside at least partially within the cavity. The process of transforming the three-dimensional digital model may include altering at least a portion of an external surface of the three-dimensional digital model, and forming a cavity within the three-dimensional digital model, wherein the cavity is sized to receive an acoustic output member and one or more drivers.

Embodiments of the present disclosure also generally relate to a custom in-ear monitor that includes an acoustic output member having a driver module that includes an output region that has an output end, wherein the output region comprises a first sound tube and a second sound tube that extend through the output region and the output end, a first driver that is coupled to the acoustic output member and is positioned to deliver an acoustic output through the first sound tube, a second driver that is coupled to the acoustic output member and is positioned to deliver an acoustic output through the second sound tube, a body and a cap that is configured to form a seal with the body when the cap is disposed over an opening and against a surface of the body. The body may include an exterior surface that is formed to substantially conform to the shape of a three-dimensional digital model that is an anatomical representation of a surface of a body part of mammal, a cavity formed within the body, wherein the cavity comprises a first region that is configured to support the output region of the acoustic output member, and a second region that is configured to enclose a portion of the first driver, the second driver and a portion of the acoustic output member, and an opening that is formed within the body and extends through the exterior surface and into the second region.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a cross-sectional view of a custom fit in-ear monitor according to an embodiment of the disclosure provided herein.

FIG. 2 is a flow chart depicting the process for manufacturing a custom fit in-ear monitor according to an embodiment of the disclosure provided herein.

FIG. 3 is a cross-sectional view of an assembly of a custom fit in-ear monitor according to an embodiment of the disclosure provided herein.

FIG. 4 is an illustration of a human outer ear and external ear canal.

FIG. 5 is a cross-sectional view of one embodiment of an optical scan device according to an embodiment of the disclosure provided herein.

FIG. 6 is a perspective view of one embodiment of a three-dimensional printing process that may be used for manufacturing a custom fit in-ear monitor according to an embodiment of the disclosure provided herein.

FIG. 7 is a cross-sectional view of one embodiment of an in-ear monitor shell according to an embodiment of the disclosure provided herein.

FIG. 8 is an illustration of a human outer ear and external ear canal with one embodiment of an in-ear monitor in place.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to a method for manufacturing a custom fit in-ear monitor. More specifically, embodiments of the present disclosure relate to a method for manufacturing a device customized to fit into the external ear canal to direct sound toward the eardrum.

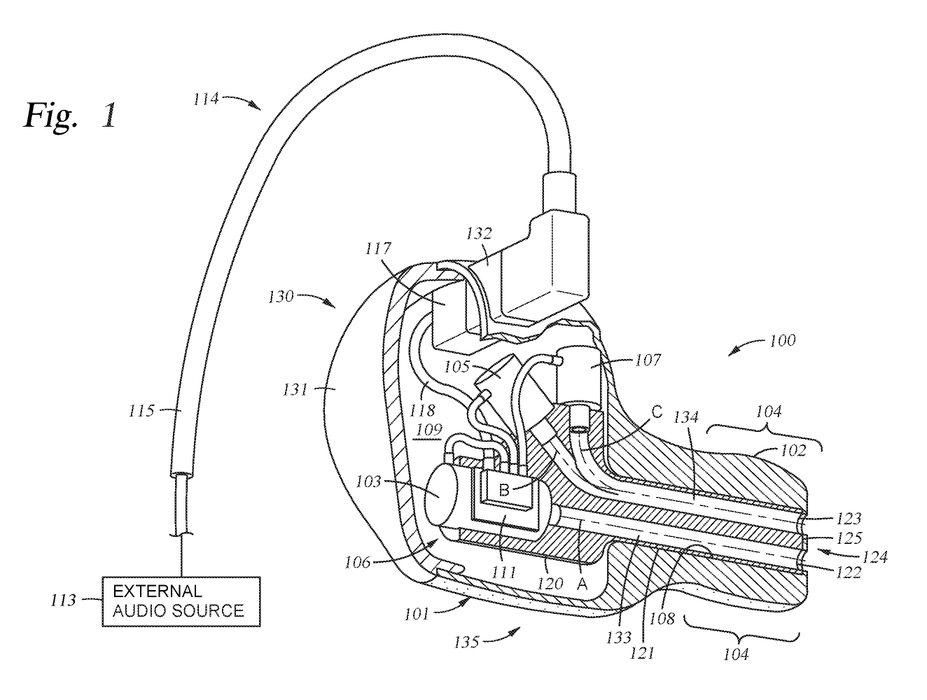

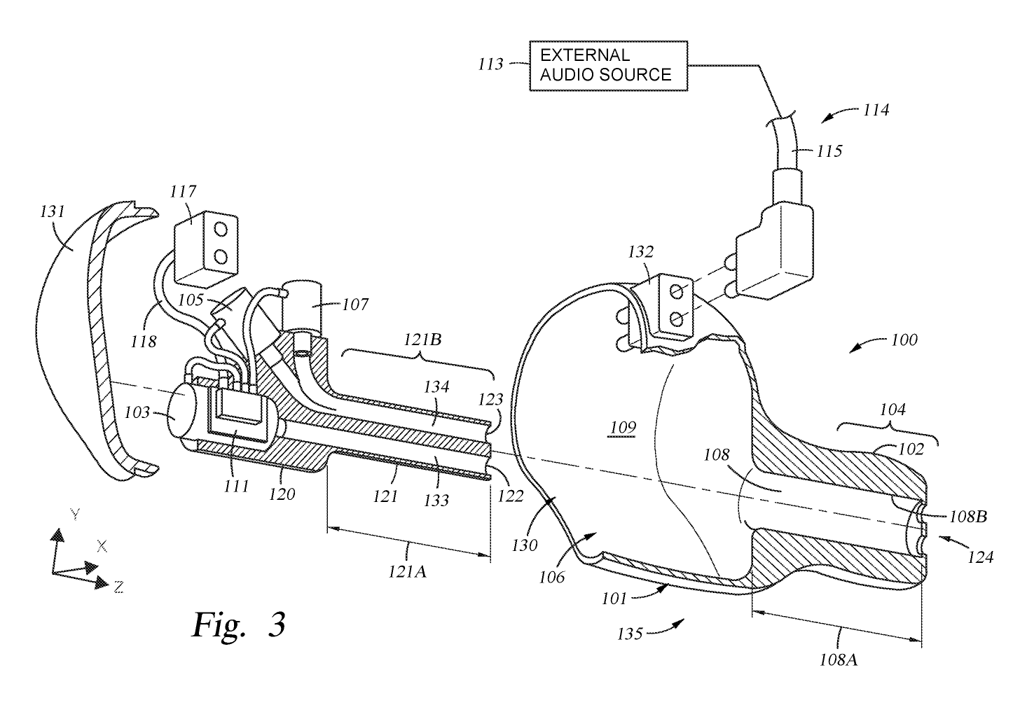

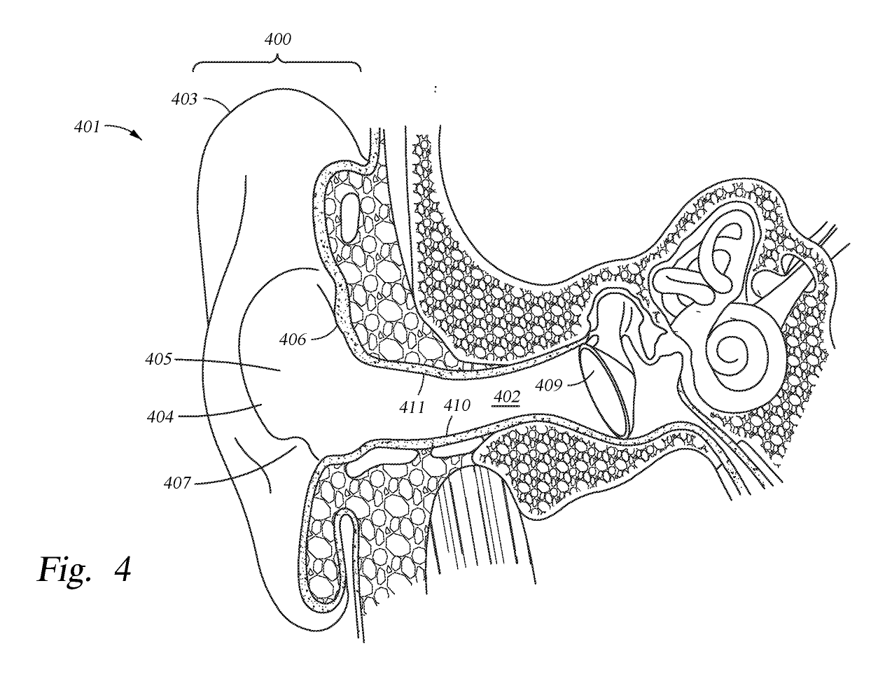

FIG. 1 is a side cross-sectional view of a custom in-ear monitor 100, according to one embodiment. Custom in-ear monitor 100 includes a custom in-ear monitor shell 101 and a removable cap 131. The custom in-ear monitor shell 101, or also referred to herein as the monitor shell body, generally includes a wall 102. The removable cap 131 is configured to fit within the opening 130 formed in the wall 102 of the in-ear monitor shell 101. In some embodiments, the removable cap 131 forms a seal with the wall 102 of the in-ear monitor shell 101. Together, wall 102 of the custom in-ear monitor shell 101 and cap 131 define a cavity 106 that has a cavity volume. The cavity volume is sized so that it can at least house a driver module 120 and a plurality of drivers. Driver module 120 includes an acoustic output member 121 which is coupled to custom in-ear monitor shell 101 at output face 124. Inner ear portion 104 of the in-ear monitor shell 101 sits inside the external ear canal 402 (FIG. 4) of the end user such that the output face 124 faces the eardrum of the end user. Inner ear portion 104 and output face 124 define a first cavity region 108 (FIGS. 1 and 3). The first cavity region 108, which is part of the cavity 106, is configured to house an output region 121B (FIG. 3) of the acoustic output member 121. A second cavity region 109 is configured to retain at least a portion of the driver module 120 and crossover circuit 111. In the embodiment shown, the driver module 120 is an assembly that includes a plurality of drivers, such as drivers 103, 105, and 107. While FIG. 1 includes three drivers 103, 105, 107, in some embodiments, the driver module 120 may comprise any number of drivers.

In one embodiment, the acoustic output member 121 of the driver module 120 includes two or more sound tubes that are formed at least partially therethrough, such as the sound tubes 133 and 134 shown in FIG. 3. The sound tubes 133 and 134 terminate at sound bores 122 and 123 at an output end 125 of the acoustic output member 121. However, the acoustic output member 121 may alternately be configured to include more than two sound tubes and sound bores or a single sound tube and sound bore. The sound tubes generally each have a length, such as distance A, distance B and distance C, as illustrated in FIG. 1. Distance A is a length of the sound tube 133 that extends from the output of the driver 103 to the output end 125 of the sound tube 133 disposed at or near the output face 124. Distance B is a length of the sound tube 134 that extends from an output of a driver 105 to the output end 125 of the sound tube 134 disposed at or near the output face 124. Distance C is a length of the sound tube 134 that extends from an output of a driver 107 to the output end 125 of the sound tube 134 disposed at or near the output face 124. In some embodiments, the driver 107 may have its own separate sound tube, and thus does not share a sound tube with driver 105, as shown in FIG. 1. Thus, in one embodiment, distances A, B and C are fixed, repeatable distances that each extend from a driver to the output end 125 of the sound bores 122 and 123. Distances A, B and C and the cross-sectional areas (e.g., circular diameters) of each of the sound tubes, such as sound tubes 133 and 134, may each be selected so that they provide reproducible high quality sound within a desired frequency range with minimal distortion. In one example, as shown, the driver 103 is coupled to output face 124 via sound bore 122, and drivers 105 and 107 are coupled to output face 124 via sound bore 123. In this example, driver 103 is preferably a high-frequency driver. In this example, driver 105 is preferably a mid-frequency driver, and the driver 107 is a low-frequency driver. In this configuration, the sound tube 133 and sound bore 122 may have a larger cross-sectional area than the sound tube 134 and sound bore 123. In some embodiments, it is desirable to fix the length 121A of the output region 121B so that the lengths (e.g., distances A, B and C) of the sound tubes can be desirably formed within driver module 120 and then the output region 121B can be repeatably and desirably positioned relative to or against the output face 124 no matter how the exterior surface of the in-ear monitor shell 101 varies in size due to the differing physical attributes of the users. In some embodiments, the length 108A of the cavity 108 is formed such that the length 108A is less than the length 121A of the output region 121B to allow the output end of the driver module 120 to be repeatably and desirably positioned relative to or against the output face 124, for all manufactured custom in-ear monitors 100 regardless of the size of the user.

The custom in-ear monitor 100 may also include a crossover circuit 111 that is either a passive crossover circuit or an active crossover circuit and provides input to the drivers 103, 105, 107 from an external audio source 113. In one embodiment, the crossover circuit 111 is electrically coupled to a cable socket 117 via cable 118, and the cable socket 117 is connected to the external audio source 113 via cable 115. Alternatively, crossover circuit 111 may be hard-wired to the in-ear monitor shell 101 via cable 118, and the in-ear monitor shell 101 may be coupled to the external audio source 113 via cable 115. Together cable 115 and external audio source 113 comprise the external assembly 114. External audio source 113 may comprise a power source (e.g., battery) and a wireless transceiver or other means for receiving user input and/or audio input from an external electronic device (e.g., mixer board, smart phone or other similar unidirectional or bidirectional audio delivery device).

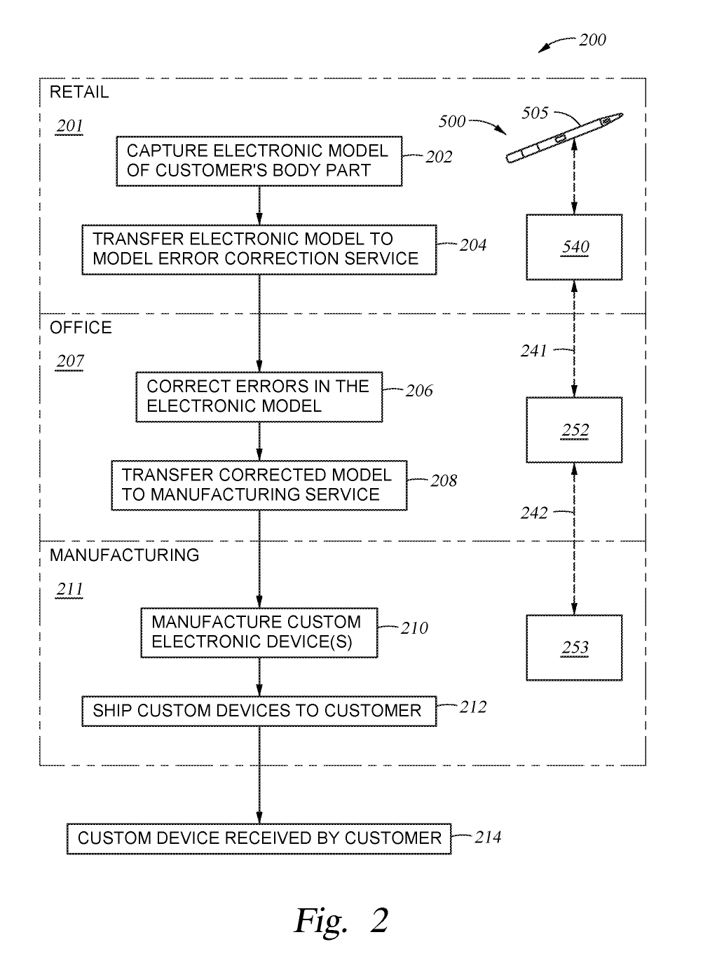

FIG. 2 is a flow chart depicting a method of manufacture 200 of a custom fit in-ear monitor 100. In operation 202, the customer's body part, such as an outer ear and external ear canal, is digitally scanned and an electronic model of the outer ear and external ear canal is created based on the scan.

For example, FIG. 4 is an illustration of a human ear and external ear canal. The outer ear 400 comprises auricle 401, which functions to collect sound and funnel it into the auditory canal or external ear canal 402. The auricle 401 comprises the helix 403 and antihelix 404. The antihelix 404 defines the concha 405 in the central part of the ear. The tragus 406 is the small bump anterior to the auditory canal 402. The antitragus 407 is the small bump below the antihelix 404. The external ear canal 402 comprises the section of the ear from the tragus 406 to the eardrum 409. The external ear canal 402 features a first bend 410 proximal to the tragus 406 and a second bend 411 proximal to the eardrum 409.

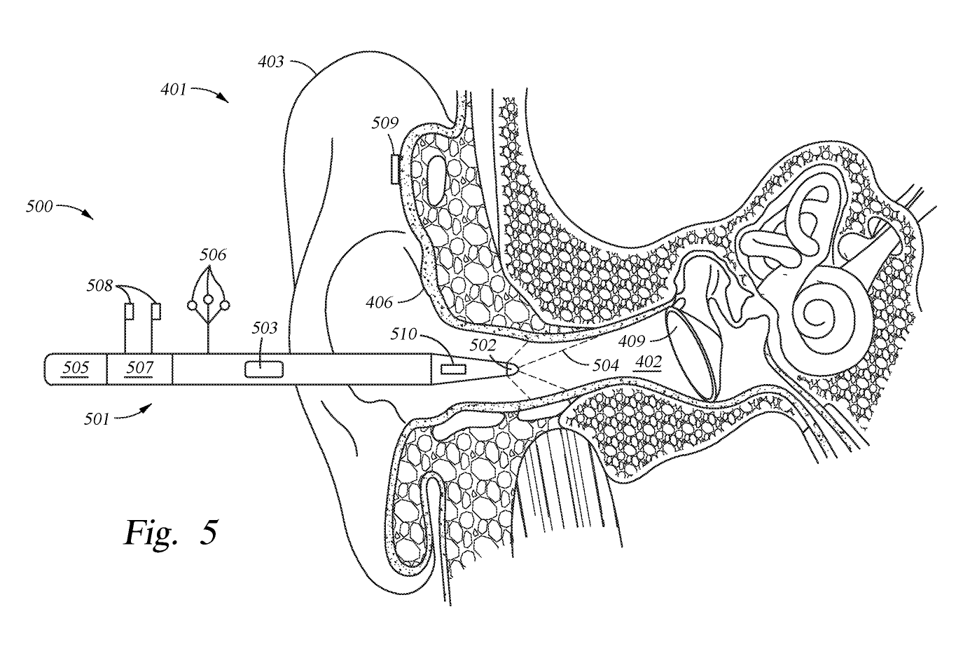

FIG. 5 is a cross-sectional view of one embodiment of a digital scan device 500. Scan device 500 may comprise a handheld scan probe 501 comprising a probe end 502 configured for insertion into or positioning near a body surface, such as an external ear canal 402 outside an eardrum 409. Probe 501 further comprises light source 503. Probe end 502 emits light 504, such as laser light, generated by light source 503. Probe end 502 further comprises light detector 505. Light detector 510 collects data relating to light 504 as it collides with the surface of ear canal 402. The collected data received by from the light detector 510 is transmitted to non-volatile memory in a first electronic device 505 by use of a processor. The processor may be any type of conventional processor that may include a central processing unit (CPU), a digital signal processor (DSP), and/or application-specific integrated circuits (ASIC), and/or other useful electrical components. Probe 501 further comprises a plurality of position detectors 506 that are in communication with the processor. Position detection system 507 comprises cameras 508, which are configured to capture images of position detectors 506 and body position detector 509. Data from position detection system 507 is transmitted to non-volatile memory in a first electronic device 505. The processor in the first electronic device 505 is configured to receive data from position detection system 507 and from light detector 505. The processor and supporting electronics within the first electronic device 505 are further configured to use the collected data to discern the dimensions and shape of a body surface such as an external ear canal 402. In some embodiments of the scan device 500, the first electronic device 505 may be connected to or be in wireless communication with an electronic device 590, as shown in FIG. 2. The electronic device 590 may be a general purpose computer that has a processor, memory and one or more communication transceivers that are able to communicate with first electronic device 505 and relay data received from the first electronic device 505 to other external electronic devices via one or more communication links.

Unlike a conventional wax mold process, the digital scan process of operation 202 is largely automated and provides an accurate rendering of the complete external ear canal 402. Therefore, the digital scan of the external ear canal 402 and outer ear 400 will not require a significant amount of skill on the part of the technician who has been trained to use scan device 500 versus a technician that is required to perform a conventional wax molding process. Therefore, operation 202 offers the advantage that it may be performed by a technician with a lower skill level and minimal training. Additionally, operation 202 does not require extensive equipment, molding supplies and a laboratory space. Instead, operation 202 requires only a scan device 500 and a technician skilled in the use of scan device 500. Therefore, operation 202 may be performed in a location separate and apart from a laboratory, such as a retail location that may be able to attract more customers (e.g., end users) or an end user's home, place of business, or location of choice. These options improve the customer experience by enhancing convenience and simplifying the scan process and ease with which the custom in-ear monitor 100 can be formed.

Returning to FIG. 2, in operation 204, the electronic model taken in operation 202 is transferred from a non-volatile memory in a first electronic device 505 of the scan device 500 at a first location to a second electronic device 252 at a second location, which is remote from the first location, and stored in a non-volatile memory therein. The scan device 500 and second electronic device are generally distinct and separate electronic devices. The transfer may be a digital transfer of electronic information such as a wired or wireless transfer of electronic data completed by transceivers positioned in the scan device 500 and the second electronic devices via a communication link 241. Operations 202 and 204 may take place at a point-of-retail 201.

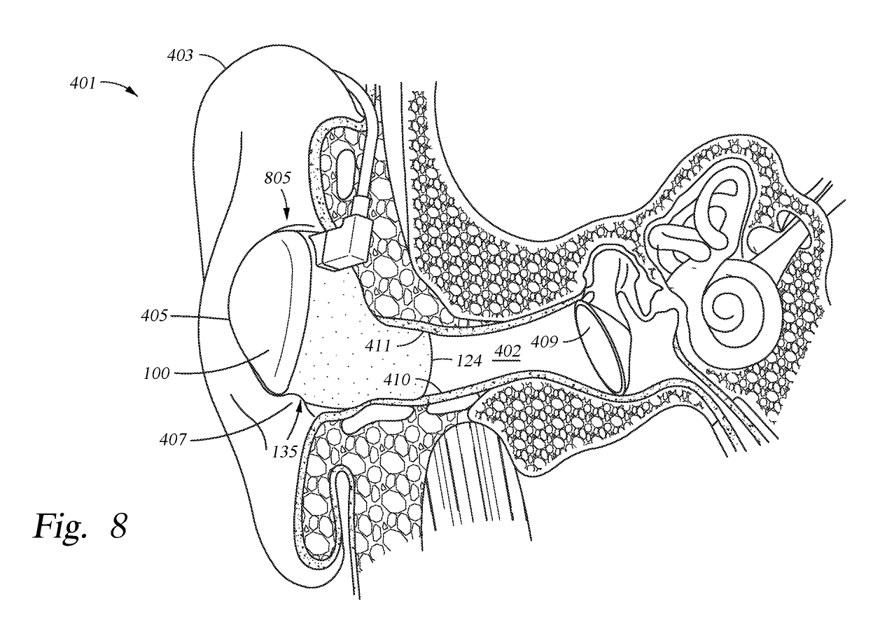

In operation 206, errors and defects in the electronic model generated by the digital scan process performed in operation 202 are corrected. During operation 206, a technician will also use the collected digital scan data of the end user's outer ear 400 to design, reconfigure and/or shape the outer ear shell portion 135, cavity 106 and/or cap 131. The technician uses data from the scan of the customer's outer ear 400 to form the shape of outer ear shell portion 135, such that the wall 102 of the in-ear monitor shell 101 will conform closely and comfortably to the shape of the customer's outer ear 400 including specifically the concha 405 (see FIG. 8). A technician further creates the cavity 106, using the data from the scan of the customer's outer ear 400 and external ear canal 402 as well as the dimensions of the driver module 120 components such that the cavity 106 can accommodate the driver module 120 and any other supporting components. A technician further designs the inner ear portion 104 of the custom in-ear monitor 100 such that the wall 102 of the inner ear portion 104 are thick enough to create a desirable fit (e.g., a location fit or slight interference fit) with at least the outer dimension of the output region 121A of the acoustic output member 121. In one example, an outer diameter of the output region 121A of the acoustic output member 121 is larger than a corresponding diameter of the cavity 108 to provide an interference type of fit. The desirable fit between acoustic output member 121 and the surface 108B of the cavity 108 of in-ear monitor 100 can be used to limit unwanted movement, vibrations and damage of the driver module 120 components during normal use. In some configurations, the driver module 120, or acoustic output member 121 portion of the driver module 120, is formed such that it has a different rigidity than the inner ear portion 104 of the custom in-ear monitor 100. In one example, the driver module 120, or acoustic output member 121 portion of the driver module 120, has a lower rigidity than of the inner ear portion 104 to allow some compression to occur in the acoustic output member 121 when it is inserted into the cavity 108 so that the driver module 120 can be desirably positionally retained due to a friction created between these parts within the custom in-ear monitor 100. The difference in rigidity between at least a portion of the driver module 120 and the inner ear portion 104 of the custom in-ear monitor shell 101 may be accomplished by the structural design of either of the components (e.g., wall thickness of each component) or by the selection of materials having differing mechanical properties. In one configuration, the driver module 120 is formed from a flexible polymeric material while the custom in-ear monitor shell 101 is formed from a more rigid polymeric material. In one example, at least a portion of the driver module 120 and the inner ear portion 104 are formed from a material such as silicone, neoprene, ethylene propylene diene monomer, nitrile rubber, nitrile, polyvinyl chloride, nitrile/PVC blends, or urethanes.

A technician may further design the cap 131 and/or the opening 130, which acts as the interface between the cap 131 and the wall 102, based on the data from the scan of the customer's outer ear 400, the shape and dimensions of the outer ear shell portion 135, and the shape and dimensions of the cavity 106. The cap 131 is designed such that the space created by the inner portion of the cap 131 and the second cavity region 109 of the cavity 106 are sized and formed to receive and accommodate the drivers 103, 105, 107, crossover circuit 111 and at least a portion of the acoustic output member 121, for example. The second cavity region 109 can be sized and formed so that it is just large enough to receive the drivers 103, 105, 107, crossover circuit 111 and at least a portion of the acoustic output member 121, which varies due to the custom size of the custom in-ear monitor shell 101. In some cases, the depth (e.g., Z-direction in FIG. 3) of the second cavity region 109 is sized so that the outer edge of the cap 131 and second cavity region 109 do not protrude outside or minimally protrude outside of the user's ear. Also, in some cases, the width dimension(s) (e.g., direction perpendicular to the axis of the cavity 108, or X and Y directions) of the second cavity region 109 are sized so that the lateral outer dimension of the custom in-ear monitor shell 101 adjacent to the second cavity region 109 are minimized and/or, for example, fits within the cavum conchae and incisura intertragica regions of the user's ear. The cap 131 and opening 130 may also be adjusted and configured to form a water-tight seal that protects the driver module 120 and its supporting components from external contamination (e.g., sweat).

In some embodiments, the length of the sound tubes 133 and 134 and dimensions of the acoustic output member 121 are fixed to a standard size for all formed custom in-ear monitors so that the acoustic properties of the sound bores 122 and 123 (e.g., diameter and length) are configured to deliver high quality sound to a user, and also has repeatable acoustic properties from one manufactured custom in-ear monitor 100 to another. In this case, the walls 102 and cavity 106 are adjusted in the custom in-ear monitor 100 to compensate for the fixed external dimensions of the driver module 120 relative to the custom shape and dimensions of the walls 102, which are adjusted to match each end user's ear. Alternately, in some embodiments, a technician may adjust the desired length of the acoustic output member 121 and properties of the sound tubes 133 and 134 based on data from the scan of the length of the customer's external ear canal 402.

In some embodiments, the output end 125 of the sound bores 122 and 123 are preferably positioned near the eardrum 409. More specifically, sound bores 122 and 123 are positioned closer to the eardrum 409 than the first bend 410 but not closer to the eardrum 409 than the second bend 411 (see FIGS. 4, 8). The determination of the lengths of sound tubes 133 and 134 dictate the positions of sound bores 122 and 123. Therefore, a technician will typically review and revise the received electronic information (e.g., data file) to refine the electronic model so that the in-ear monitor shell 101 can be desirably formed in subsequent steps. To do so, a technician reviews the data file and makes changes to the data file as necessary to form the wall 102 of the in-ear monitor shell 101. For example, the data file as scanned and provided may not be in a format that may be easily formed by an additive manufacturing process, such as a three-dimensional printing process. In this case the technician may make changes to the data file so that it may be readily printed. In another example, the data file may capture a shape of a formed in-ear monitor shell 101 that may not be easily inserted into or removed from the external ear canal 402 because of the individual's particular ear physiology. In this case, the technician may revise the data file such that the formed shell may be easily inserted and removed given the user's ear physiology. In yet another example, the technician must design the cavity 106 of the formed in-ear monitor shell 101 such that the outer in-ear monitor shell 101 fits comfortably into the user's outer ear 400, while the inner portion of the in-ear monitor shell 101 accommodates the driver module 120. In yet another example, as noted above, the technician alters the design of the cavity 106 such that the cap 131 fits into the edge of a portion of the walls 102 to create a seal, such that sweat or other elements may not enter the cavity 106 and compromise the driver module 120. In yet another example, the technician may smooth out the exterior surface of the wall 102 in the electronic model so that the formed custom in-ear monitor 100 will be more comfortable for the user during normal use. The technician may smooth out a region of the electronic model so that the surface in the “smoothed region” has a more even and regular surface, such that the region is free from perceptible projections, roughness, sharp edges and/or indentations.

In operation 208, the corrected electronic model formed in operation 206 is transferred from a non-volatile memory in a second electronic device 252 at a second location to a non-volatile memory in a third electronic device 253 at a third location remote from the second location. Second electronic device 252 and third electronic device 253 are distinct electronic devices. The transfer may be a digital transfer such as a wired or wireless transfer of electronic data via a communication link 242. Operations 206 and 208 may take place at an office 207, which is different from and/or a distance from the point-of-retail 201. In some embodiments, the office 207 is positioned in an area that has a lower rent and/or real property value than the point-of-retail 201, and thus, in some examples, may be across the street, across the country or across the world from the point-of-retail 201 location.

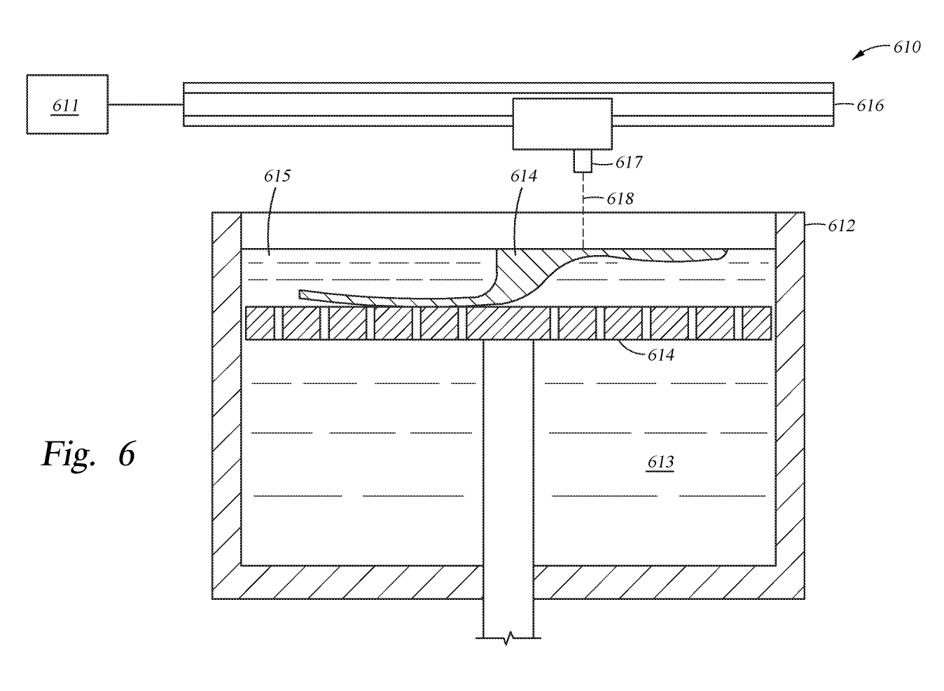

In operation 210, the custom in-ear monitor(s) 100 are manufactured. In operation 210 the outer in-ear monitor shell 101 is formed using an additive manufacturing process, such as a printing process described below in conjunction with FIG. 6. In operation 210 the assembly of the custom in-ear monitor 100 is completed. FIG. 3 is an exploded cross-sectional view of the custom in-ear monitor 100 assembly. After the elements of custom in-ear monitor 100 are refined and printed, the drivers 103, 105, 107, crossover circuit 111 and acoustic output member 121 are inserted into the cavity 106, 108 of the formed outer in-ear monitor shell 101, such that the acoustic output member 121 is directed toward the output face 124 with sound bores 122, 123 proximal to output face 124. After positioning driver module 120 into cavity 106, cavity 106 is sealed with the insertion of the cap 131 in the opening 130 to protect driver module 120. Cap 131 is then fitted over driver module 120 to create a seal and protect driver module 120. Driver module 120 fits closely into in-ear monitor shell 101 such that sound bores 122, 123 are flush with the end of output face 124. This arrangement allows for close placement of the drivers 103, 105, and 107 to the eardrum 409 to improve sound quality, noise isolation and to prevent feedback. After sealing the cavity 106, the electrical components of the driver module 120 are electrically connected to an external audio source 113. Alternatively, the electrical components of the driver module 120 may be electrically connected to external audio source 113 before sealing cavity 106 in order to more easily connect or test the effectiveness of the driver module 120.

Operation 210 may occur at a location separate and apart from the location at which operations 206 and/or 208 take place. For example, while a skilled technician may be required to perform operation 206, special tools and machinery (such as three-dimensional printers) and less skilled technicians may be required to perform operation 210. Therefore, operation 206 may take place in the office 207 (e.g., office building), while operation 210 may take place in a warehouse where three-dimensional printers and their supporting materials are stored. This arrangement may allow for cost savings in that the large machinery and materials may be maintained in a less expensive location than the office 207 and/or point-of-retail 201. However, in some embodiments, the processes performed in operation 210 may occur at the original point-of-retail 201 so that the end user can easily pick-up the completed custom in-ear monitor 100.

In operation 212, the custom device or devices are shipped to the customer. Operations 210 and 212 take place at manufacturing facility 211. In operation 214, the customer receives the complete custom in-ear monitor device 100.

### Additive Manufacturing Process Example

FIG. 6 is a cross-sectional view of one embodiment of an additive manufacturing process, such as a three-dimensional printing process, that may be used during operation 210 to at least manufacture the outer in-ear monitor shell 101. One example of a three-dimensional printing method that may be used for printing parts of the custom in-ear monitors 100 is a stereolithography formation process. Stereolithography is capable of rapidly forming small detailed parts. In the stereolithography formation process, software is used to digitally divide a three-dimensional model of the item to be printed (e.g., corrected electronic model) into multiple horizontal layers. The sliced model 611 is delivered to the three-dimensional printer 610. The three-dimensional printer 610 includes a tub 612 containing liquid resin 613. A build platform 614 rests at or near the surface 615 of the liquid resin 613. The image projection module 616 comprises a UV laser 617 that can be scanned over a portion of the build platform 614 that is disposed within the liquid resin 613. The UV laser 617 projects a laser beam 618 to form an image of the layer to be formed within a portion of the liquid resin 613. A controller within the printer 610 causes the laser beam 618 to trace the pattern of the single layer 619 onto the surface 615 of the liquid resin 613. Exposure to the laser beam 618 cures the liquid resin 613, solidifying a single layer 619 of resin on the build platform 614. After single layer 619 is deposited, build platform 614 lowers incrementally and subsequent layers are added by curing the liquid resin 613 and fusing it to existing layer 619 until the full model is built. Once the model is built, build platform 614 supporting the built model 620 rises out of the liquid resin 613. The built model 620 is then cleaned, any added supports are removed, and the model is further UV cured. Examples of three-dimensional printers that may be used for manufacturing custom fit in-ear monitors include Projet 6000 by 3D Systems and Fab13 by Pro3dure. These three-dimensional printers are appropriate for printing customized in-ear monitors because these printers are capable of printing small items in great detail, and because they yield relatively smooth junctions between adjacently formed layers. However, the exterior surface of the wall 102 may also be further buffed in a post process, as described below. One will appreciate that other types of three-dimensional printers may be used to form a custom in-ear monitor.

After the in-ear monitor shell 101 is formed, cleaned and cured, additional processing may take place during operation 210. For example, because printing process 600 involves the deposition of layers of resin, the process yields a built model 620 that may have an imperfect, ridged surface. For user comfort, the ridges must be smoothed by reducing variations in the surface roughness of the in-ear monitor shell 101. Therefore a technician must smooth the outer surface of the in-ear monitor shell 101 after the printing process has concluded.

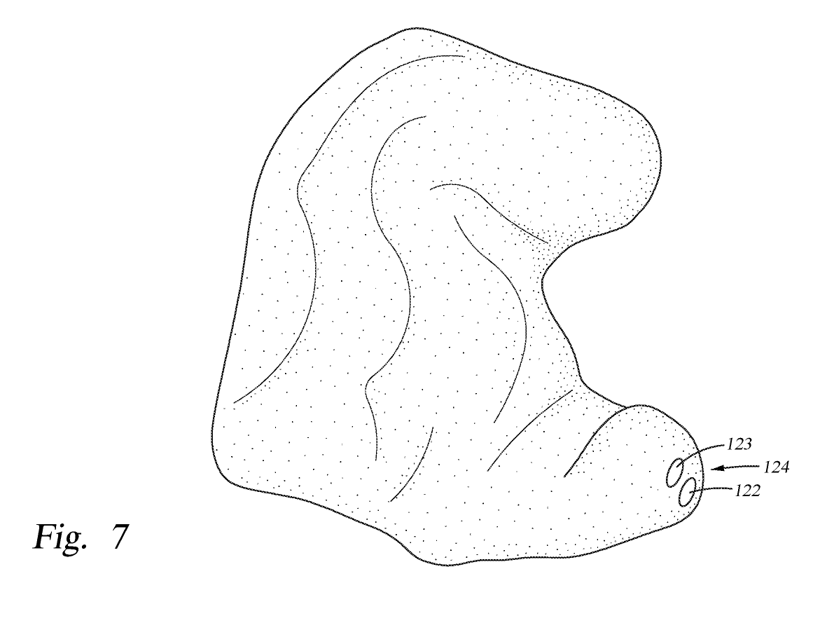

FIG. 7 is a perspective view of one embodiment of an outer in-ear monitor shell 101, which is intended to illustrate the custom and smoothed exterior shape of the wall 102 after being formed. Once completed, the in-ear monitor shell 101 is designed to fit closely and comfortably mate with the user's body part that was digitally scanned.

FIG. 8 is an illustration of a human outer ear and external ear canal with one embodiment of an in-ear monitor 100 in place. In-ear monitor 100 fits snugly into outer ear 400 and external ear canal 402 such that in-ear monitor 100 cannot be easily dislodged or moved. In-ear monitor 100 is positioned in the external ear canal 402 such that output face 124 and sound bores 122 and 123 are disposed between first bend 410 and second bend 411. Outer ear shell portion 124 fits closely against concha 405 and antihelix 404 and may contact tragus 406 and/or antitragus 407 to allow for comfort and to reduce movement of in-ear monitor 100.

### Additional Manufacturing Process Example

By eliminating the necessity of tuning each in-ear monitor (IEM) 100 prior to completion of the custom in-ear monitor 100, due to the presence of the optimized and standardized configuration of the acoustic output member 121, embodiments of the present disclosure allow the IEM manufacturing process to be substantially altered from the traditional, more labor intensive processes typically used to manufacture custom-fit IEMs. In one example, the manufacturing process includes after an end user's ear is molded using a conventional wax molding technique to form an ear mold, the ear mold itself is digitally scanned, for example using a three-dimensional (3D) scanner, in order to create a data file that represents the shape of the desired ear mold. The data file is then analyzed and modified to create a final data file that represents the desired external shape as well as the desired internal features that will allow the ear mold to accommodate the driver module 120 and drivers 103, 105 and 107. Using the modified data file, a 3D printer is then used to fabricate the in-ear monitor shell 101. Once the in-ear monitor shell 101 is fabricated and the drivers 103, 105, and 107, and crossover circuit 111 have been installed onto the driver module, the acoustic output member 121, drivers 103, 105, and 107, and crossover circuit 111 are inserted into the in-ear monitor shell 101 and the in-ear monitor shell 101 is sealed in order to protect the internal components of the custom in-ear monitor 100.

As a result of simplifying the manufacturing and assembly process, the improved process allows portions of the process to be performed remotely and off-site. For example, the ear mold may be made and scanned at a first location convenient for the end user, for example a store within a shopping mall, a stand-alone store, or a region carved out of an existing store (e.g., a store-within-a-store). The data file created at the first location can then be sent to another site, for example a central processing site (e.g., second location) in a different geographic region, for analysis. At the central processing site, the initial data file is analyzed and modified to include the desired internal features that will allow the ear mold to accommodate the driver module 120. The final data file along with assembly instructions are then sent back to the remotely located store (e.g., first location) where the in-ear monitor shell 101 is fabricated, for example using a 3D printer. The driver module 120, i.e., acoustic output member 121, drivers 103, 105, and 107, and crossover circuit 111, is then assembled and inserted into the in-ear monitor shell 101 after which the in-ear monitor shell 101 is sealed by the insertion of the cap 131.

While the foregoing is directed to embodiments of the present disclosure, other and further embodiments of the disclosure may be devised without departing from the basic scope thereof, and the scope thereof is determined by the claims that follow.

What is claimed is:

1. A method of forming a custom in-ear monitor, comprising:
capturing a digital anatomical representation of a surface of a body part at a first location, wherein capturing comprises digitally scanning at least a portion of the body part and storing data associated with the captured surface dimensions of the body part in non-volatile memory of a first electronic device;
transferring the stored data to a second electronic device positioned at a second location;
forming a three-dimensional digital model from the stored data using the second electronic device;
transforming the three-dimensional digital model, wherein the transforming comprises:
altering at least a portion of an external surface of the three-dimensional digital model, and
forming a cavity within the three-dimensional digital model, wherein the cavity is sized to receive an acoustic output member and one or more drivers;
transferring the transformed three dimensional model to a third electronic device positioned at a third location;
forming, at the third location, a monitor shell of an in-ear monitor using the transformed three dimensional model; and
positioning the acoustic output member and one or more drivers within the formed monitor shell, wherein the acoustic output member and one or more drivers reside at least partially within the cavity.

2. The method of claim 1, wherein the first and third locations are the same location.

3. The method of claim 1, wherein the acoustic output member and one or more drivers are adapted to connect to an audio source.

4. The method of claim 3, wherein the audio source comprises a wireless transceiver.

5. The method of claim 3, wherein the audio source comprises means for receiving user input.

6. The method of claim 1, wherein the second location is physically remote from the first location such that the second location has a lower rent or real property value than the first location.

7. The method of claim 6, wherein the third location is physically remote from the second location.

8. The method of claim 1, wherein the third location is physically remote from the second location.

9. The method of claim 1, wherein the body part is a human outer ear and external ear canal.

10. The method of claim 1, wherein forming the monitor shell using the transformed three dimensional model comprises using an additive manufacturing process to form the monitor shell.

11. The method of claim 10, wherein the additive manufacturing process comprises three-dimensional printing method that comprises a stereolithography process.

12. The method of claim 1, further comprising:
after positioning the acoustic output member and one or more drivers within the formed monitor shell, sealing the cavity with a cap.

13. The method of claim 1, further comprising:
after forming a monitor shell of an in-ear monitor using the transformed three dimensional model, and
cleaning the formed monitor shell.

14. The method of claim 1, further comprising:
after forming a monitor shell of an in-ear monitor using the transformed three dimensional model, and
reducing the surface roughness of the formed monitor shell.

15. A custom in-ear monitor, comprising:
an acoustic output member having a member body that includes an output region that has an output end, wherein the output region comprises a first sound tube and a second sound tube that extend through the output region and the output end;
a first driver that is coupled to the acoustic output member and is positioned to deliver an acoustic output through the first sound tube;
a second driver that is coupled to the acoustic output member and is positioned to deliver an acoustic output through the second sound tube;
a monitor shell body comprising:
an exterior surface that is formed to substantially conform to the shape of a three-dimensional digital model that is an anatomical representation of a surface of a body part of mammal;
a cavity formed within the monitor shell body, wherein the cavity comprises:
a first region that is configured to support the output region of the acoustic output member; and
a second region that is configured to enclose a portion of the first driver, the second driver and a portion of the acoustic output member; and
an opening that is formed within the monitor shell body and extends through the exterior surface and into the second region; and
a cap that is configured to form a seal with the monitor shell body when the cap is disposed over the opening and against a surface of the monitor shell body.

16. The custom in-ear monitor of claim 15, wherein the exterior surface includes one or more regions that differ from the three-dimensional digital model, wherein the one or more regions are smoother than the equivalent portion of the three-dimensional digital model.

17. The custom in-ear monitor of claim 15, wherein the first sound tube and the second sound tube each have a different cross-section area.

18. The custom in-ear monitor of claim 15, wherein the monitor shell body further comprises an output face that is positioned to face an eardrum when in use, wherein the output end is disposed proximate to the output face.
